(19) United States
(12) Patent Application Publication
(10) Pub. No.: US 2020/0015912 A1
(43) Pub. Date: Jan. 16, 2020

(54) SURGICAL INSTRUMENT OF REMOVABLE TIP TOOL AND METHOD

(71) Applicant: Luiz Lanat Pedreira de Cerqueira Filho, Orlando, FL (US)

(72) Inventor: Luiz Lanat Pedreira de Cerqueira Filho, Orlando, FL (US)

(21) Appl. No.: 16/505,317

(22) Filed: Jul. 8, 2019

Related U.S. Application Data
(63) Continuation-in-part of application No. 16/458,216, filed on Jul. 1, 2019.
(60) Provisional application No. 62/748,562, filed on Oct. 22, 2018.
(60) Provisional application No. 62/735,169, filed on Sep. 23, 2018.
(60) Provisional application No. 62/695,139, filed on Jul. 8, 2018.
(60) Provisional application No. 62/692,793, filed on Jul. 1, 2018.

(51) Int. Cl. A61B 34/20; A61B 34/30; A61B 17/29; A61B 90/00

(52) U.S. Cl. CPC A61B 34/20; A61B 34/30; A61B 2017/00477; A61B 90/361; A61B 90/37; A61B 17/29

(57) ABSTRACT

The inventor provides a surgical device that performs a video-surgery without removing the video-surgical instrument out of the surgical site and a method of use. The invention including: an elongated body; said body including a proximal end and a distal end; a tip tool connectable to said distal end; and a handle connectable to said proximal end; wherein said distal end is adapted to interchangeably connect to said tip tool inside a surgical site. In some embodiments said tip tool is a toolbox. Methods of using the toolbox in a surgery are provided.

### CROSS-REFERENCE TO RELATED APPLICATIONS

This application claims an invention which was disclosed in a provisional application No. 62/695,139, filled in Jul. 8, 2018 entitled “Surgical Instrument of Removable Tip Tool and method”, and in part of the nonprovisional application Ser. No. 16/458,216, filled in Jan. 7, 2019 entitled “Free scar instrument and method”. The benefit under 35 USC § 119(e) of the United States provisional application is hereby claimed, and the afore mentioned application is hereby incorporated herein by reference.

### DESCRIPTION

### Field of Invention

The invention is in the field of medical devices and pertains, particularly, to an instrument for video-surgery including an interchangeably tip tool and method of use.

### Background

In the art of medical devices for performing video-surgery, many different types of video-surgical instruments have been developed to perform video-surgery.

One problem with the traditional video-surgical instruments is during the operative act the instruments of video-surgery need to be constantly removed from the operative surgical site 11 and replaced by others that are being needed in the surgery at that moment. For example, the surgeon initiates surgery using tweezers and then needs to use scissors. Another limitation of the current video surgical instruments the surgeon needs to remove the tweezers from the inside of a trocar, delivers it to his assistant, takes the scissors in the assistant's hand, places the scissors in the trocar, and resumes operation. Another limitation of the current video surgical instruments, these movements take time, increase the risk of contamination from surgery and are dangerous.

Minimally invasive surgical devices for performing surgery, such as: robotic surgery, laparoscopic video surgery, thoracoscopic video surgery, video cardiac surgery, video arthroscopic surgery, video urologic surgery, video neurological surgery, video ophthalmologic surgery, orthopedic video surgery, and others video-surgical instruments need to be removed from the surgical side during the surgery.

The possibility of performing surgery without removing the video-surgical instrument from the operative site is a grate evolution of surgery. The great advantage is the reduction of the surgical time, the infection and the survival of the patient. Surgery becomes faster, more efficient and safer.

Therefore, what is clearly needed is a surgical device that performs the surgery without removing the video-surgical instrument out of the surgical site and a method of use that solves the problems mentioned above.

### SUMMARY OF THE INVENTION

In one embodiment of the invention, an instrument for video-surgery, comprising: an elongated body; said body including a proximal end and a distal end; a tip tool connectable to said distal end; and a handle connectable to said proximal end; wherein said distal end is adapted to interchangeably connect to said tip tool inside a surgical site, is provided.

Also, in another embodiment, said handle is in functional connection to said proximal end for the transmission of at least one of force, power and torque, to said tip tool, is provided.

Also, in another embodiment, said handle is adapted to interchangeably connect to a plurality of external apparatus for the transmission of at least one of force, power and torque, to said tip tool, is provided.

Also, in another embodiment, further including an extension connectable do said distal end and connectable to said tip tool, is provided.

Also, in another embodiment, further including at least one articulation joint, is provided.

Also, in another embodiment, said distal end is adapted to interchangeably connect to a plurality of said extensions inside said surgical site, is provided.

Also, in another embodiment, said handle is adapted to be robotic operating, is provided.

Also, in another embodiment, said tip tool includes a toolbox, is provided.

Also, in another embodiment, said tip tool includes a pair of cooperating jaws made to perform a surgical operation, is provided.

Also, in another embodiment, said tip tool is rotatable around one or more axes, is provided.

In another embodiment of the invention, a method of performing a video-surgery, said method comprising the steps of: (a) inserting a video-surgical instrument inside a surgical site through the trocar; (b) interchanging a tip tool of said video-surgical instrument to another said tip tool inside said surgical site; and (c) performing said video-surgery with said video-surgical instrument, is provided.

The method, further comprising connecting said tip tool to said video-instrument inside said surgical site, is provided.

The method, further comprising disconnecting said tip tool of said video-instrument inside said surgical site, is provided.

The method, further comprising inserting the toolbox inside said surgical site through another said trocar, is provided.

The method, further comprising connecting at least one said tip tool to said toolbox, is provided.

The method, further comprising using said toolbox to interchange said tip tool of said video-instrument with another said tip tool inside said surgical site, is provided.

The method, further comprising inserting another said video-surgical instrument caring one or more tip tools inside said surgical site through another said trocar, is provided.

The method, further comprising connecting an external apparatus to said video surgical instrument, is provided.

The method, further comprising interchanging said external apparatus to another said external apparatus, is provided.

The method, further comprising interchanging said tip tool to another said tip tool inside said surgical site, is provided.

In another embodiment of the invention, a method of using the toolbox in a video-surgery, said method comprising the steps of: (a) disconnect a tip tool of a video-surgical instrument and connect to said toolbox; (b) choose another said tip tool in the toolbox; and (c) disconnect said tip tool of said toolbox and connect said tip tool to said surgical instrument, is provided.

The method, further comprising connecting said toolbox to a video-surgical instrument, is provided.

The method, further comprising inserting said toolbox connected to said video-surgical instrument in said surgical site through the trocar, is provided.

The method, further comprising inserting at least one tip tool in said toolbox, is provided.

The method, further comprising disconnecting said toolbox of said surgical instrument and connecting said toolbox to another video instrument inside said surgical site, is provided.

The method, further comprising inserting another video-surgery instrument into said surgical site through another said trocar, is provided.

The method, further comprising, inserting an extension in said toolbox, is provided.

The method, further comprising using said toolbox to connect an extension in said video-surgical instrument, is provided.

The method, further comprising removing said toolbox connected to said video-surgical instrument out of said surgical site through said trocar, is provided.

The method, further comprising disconnecting said toolbox of said video-surgical instrument, is provided.

The method, further comprising using said toolbox to connect said tip tool in said surgical instrument inside said surgical site, is provided.

The method further comprising connecting using said toolbox to disconnect said tip tool of said surgical instrument inside said surgical site, is provided.

The method further comprising using said toolbox to save at least one of said tip tool, is provided.

### BRIEF DESCRIPTION OF THE DRAWINGS

FIG. 1 is a perspective view of the current invention in which the adapter 2 is engaged in the tip tool 1 forming a single set, according to one embodiment of the invention.

FIG. 2 is a perspective view of one embodiment of the distal end 3B, the adapter 2 and the tip tool 1, according to another embodiment of the invention.

FIG. 3 is a perspective view of the distal end 3B of the present instrument, the body 3 is sectioned and viewed from its distal end 3B, according to another embodiment of the invention.

FIG. 4 is a view of the invention wherein tip tool 1 is the toolbox 7, according to one embodiment of the invention.

FIG. 5 is a representation of the use of the present invention, according to another embodiment of the invention.

FIG. 6 is a perspective view of the tip tool 1 which is a tweezer with a jaw 10A and jaw 10B closed, according to another embodiment of the invention.

FIG. 7 is a perspective view of the adapter 2 and the distal end 3B of the body 3, according to another embodiment of the invention.

FIG. 8 is a perspective view of two video-surgical instruments, in accordance with other embodiment of the invention.

FIG. 9 is another perspective view of two video-surgical instruments showing the toolbox 7 connected to four tip tool 1, in accordance with another embodiment of the invention.

FIG. 10 is a perspective view of a video-surgical instrument that has just replaced tip tool 1 with another tip tool 1 using the toolbox 7, according to another embodiment of the invention.

### DETAILED DESCRIPTION OF THE PREFERRED EMBODIMENTS

The inventor provides a surgical device that performs the surgery without removing the video-surgical instrument out of the surgical site and methods of use. The invention is described in enabling detail in the following examples, which may represent more than one embodiment of the invention, together with the accompanying drawings in which like numerals represent similar components. Additionally, the structures described herein can be embodied as integrated components or as separate components.

In one characteristic of this embodiment, the invention seeks to provide a solution to this problem by providing a surgical instrument of removable tip tool 1. In one characteristic of some embodiment, the invention allows performing the video-surgery, with minimal trauma, without the need to remove the instrument from the surgical site 11, the tip tool 1 is easily replaced within the surgical site. In one characteristic of some embodiment of the invention, the tip tool 1 is easily removable from an adapter 2 of the instrument and replaced with another tip tool 1 inside the surgical site 11 with the aid of a toolbox 7.

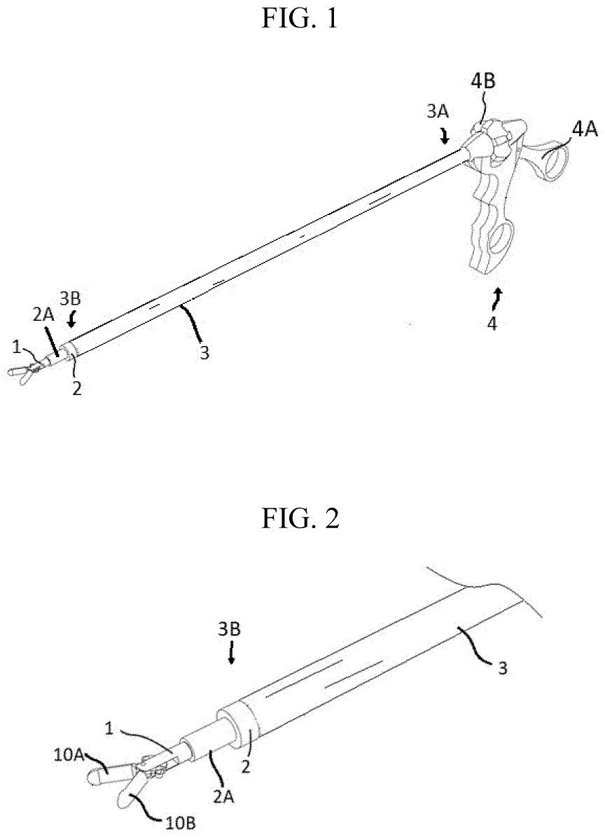

FIG. 1 is a perspective view of the current invention in which the adapter 2 is engaged in the tip tool 1 forming a single set, according to another embodiment of the invention.

In one characteristic of this embodiment, it includes an elongated body 3; said body 3 including the proximal end 3A and the distal end 3B; the tip tool 1 is connectable to said distal end 3B; and a handle 4 is connectable to said proximal end 3A; wherein said distal end 3B is adapted to interchangeably connect to said tip tool 1 inside a surgical site.

In one characteristic of this embodiment, the handle 4 is in a functional connection to said proximal end 3A for the transmission of at least one of force, power and torque, to said tip tool 1. In some embodiments, the handle 4 is adapted to interchangeably connect to a plurality of external apparatus for the transmission of at least one of force, power and torque, to said tip tool 1.

In one characteristic of this embodiment, the tip tool 1 may be rotated relative to the body 3 longitudinal axis from the rotation of a piece 4B. The piece 4B is attached to a stem 3C which is connectable to a twirl 2A. In one characteristic of this embodiment, the opening and closing of the lever 4A cause longitudinal movement of the stem 3C, this movement opens or closes the jaw 10A and jaw 10B, according to another embodiment of the present invention.

In another characteristic of some embodiments, the rotation mechanism of the twirl 2A is adapted to be done by the rotation of the stem 3C or by an electric motor in the adapter 2, with gear, but is not limited to them.

In one characteristic of some embodiments said tip tool 1 is rotatable around one or more axes.

In one characteristic of some embodiments said tip tool 1 is the toolbox 7.

In one characteristic of some embodiments said tip tool 1 comprises a pair of cooperating jaws 10 made to perform a surgical operation.

FIG. 2 is a perspective view of one embodiment of the distal end 3B, the adapter 2 and the tip tool 1, according to another embodiment of the invention. In one characteristic of this embodiment, the adapter 2 is fitted into the tip tool 1 forming a single set. In one characteristic of this embodiment, the tip tool 1 may be rotated relative to the longitudinal axis of the body 3 from the rotation of the Piece 4B. In one characteristic of this embodiment, the stem 3C is engaged with the tip tool 1, rotation of the stem 3C causes the tip tool 1 to rotate. In one characteristic of this embodiment, the opening and closing of the lever 4A cause longitudinal movement of the stem 3C, this movement opens or closes the jaw 10A and jaw 10B. According to another embodiment of the present invention.

In another characteristic of some embodiments, the invention is adapted to be made to work with groups of different tip tool 1, for example: scissors, mixer, clamp and other tip tool 1 which do not have an electric mechanism; stapler with electric mechanism, ultrasonic scissors, but not limited to them. In another characteristic of some embodiments, when using an electric mechanism, appropriate connectors, wires, switches and fittings are used but not limited to them.

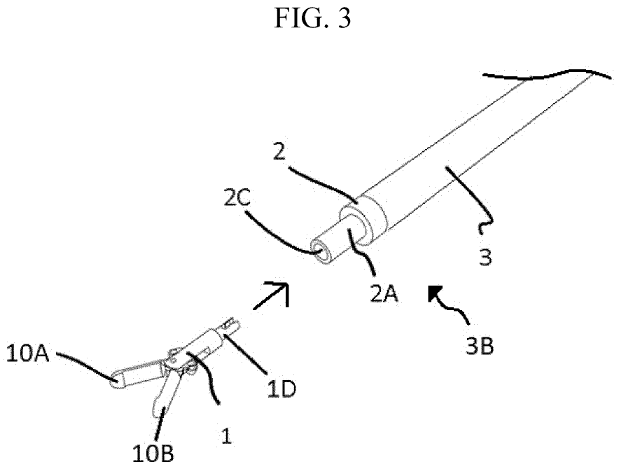

FIG. 3 is a perspective view of the distal end 3B of the present instrument, the body 3 is sectioned and viewed from its distal end 3B, according to one embodiment of the invention. In one characteristic of this embodiment, a pin 1D is uncoupled from a fitting 2C. In one characteristic of this embodiment, the jaw 10A and jaw 10B are open. In one characteristic of this embodiment, the arrow points to the direction that tip tool 1 should follow to fit into the fitting 2C of the adapter 2, according to another embodiment of the present invention.

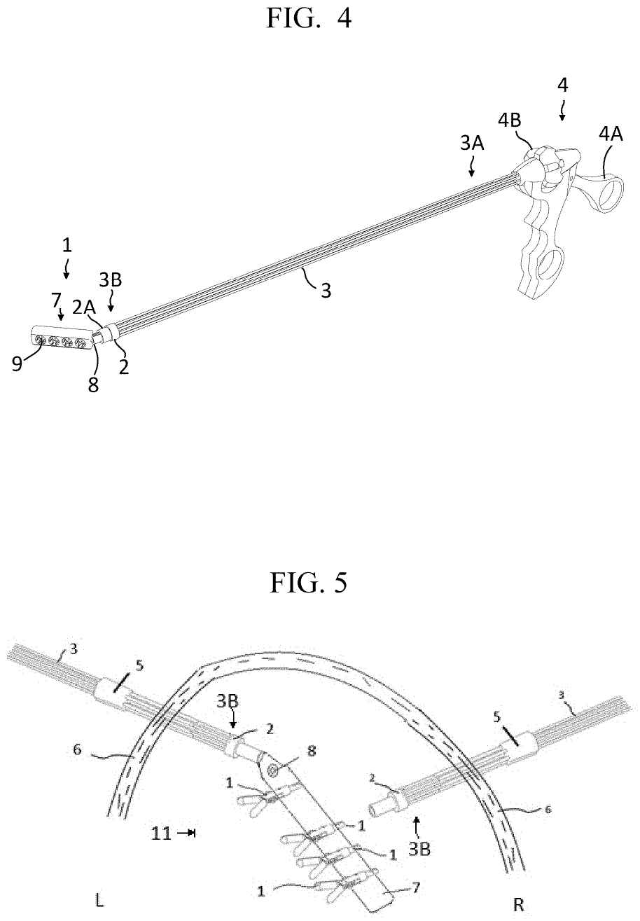

FIG. 4 is a view of the invention wherein tip tool 1 is the toolbox 7, according to one embodiment of the invention. This embodiment includes the articulation joint 8. The articulation joint 8 is an angulation mechanism, the opening and closing of lever 4A causes tip tool 1 to change its angle relative to the longitudinal axis. In one characteristic of this embodiment, the rotation of Piece 4B causes twirl 2A to rotate relative to the longitudinal axis, the toolbox 7 rotates with twirl 2A. A socket 9 is where tip tool 1 fits. Four socket 9 are seen for four tip tool 1 docking, according to another embodiment of the present invention.

FIG. 5 is a representation of the use of the present invention, according to another embodiment of the invention. Two video-surgical instruments of removable tip tool 1 are seen, one on the right R and the other on the left L side, each passing a trocar 5. In one characteristic of this embodiment, two trocar 5 are crossing the wall of a tissue 6. In one characteristic of this embodiment, the distal end 3B of the two instruments are inside the surgical site 11. On the left side L, the tip tool 1 including the toolbox 7 is embedded in the adapter 2 forming a single set. Four tip tool 1 are in the toolbox 7. The toolbox 7 is angled by the articulation joint 8 with four available tip tool 1 to be chosen and fitted into the adapter 2 of the invention, which is on the right-side R, according to another embodiment of the present invention.

In another characteristic of some embodiments, the toolbox 7 is inserted into the surgical site 11 through another trocar 5, connected in the adapter 2 of another video-surgery instrument. Generally, after insertion in the surgical site, the toolbox 7 is positioned in an easily accessible location in the surgical site 11.

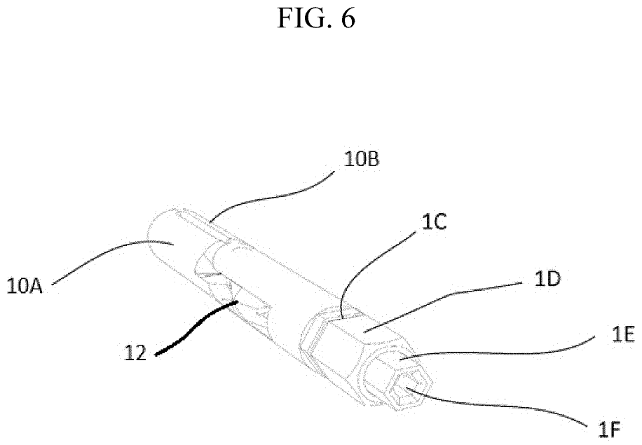

FIG. 6 is a perspective view of the tip tool 1 which is a tweezer with a jaw 10A and jaw 10B closed, according to one embodiment of the invention. It is seen a crevice 1C where a locks 2B fits. The pin 1D is made ready to fit into the fitting 2C. A link 1E is attached to the jaw 10A and jaw 10B joint 12. In one characteristic of this embodiment, the longitudinal movement of the link 1E opens and closes jaw 10A and jaw 10B. The Lumen 1F is where the stem 3C fits removably, according to another embodiment of the invention.

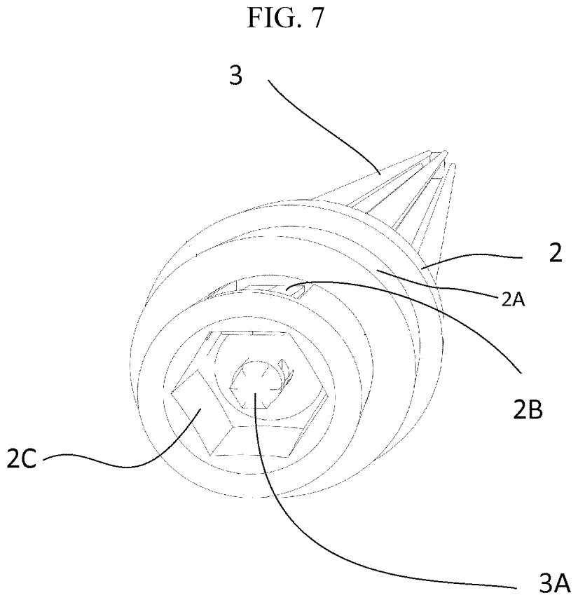

FIG. 7 is a perspective view of the adapter 2 and the distal end 3B of the body 3, according to another embodiment of the invention. The twirl 2A rotates by the action of stem 3C, locks 2B is a lock securing the pin 1D in the fitting 2C, locks 2B fits in the crevice 1C. Locks 2B is triggered by the handle 4 mechanism or by electromagnetic action when locks 2B is inserted into a socket 9 of the toolbox 7. The fitting 2C is where the pin 1D fits. The fitting 2C in this embodiment features hexagonal shape.

In another characteristic of some embodiments, the tip tool 1 is attached to the adapter 2 by the locking mechanism locks 2B. In another characteristic of some embodiments, the locks 2B is drive by the stem 3C which is moved from a lever 4A in the handle 4. In another characteristic of some embodiments, locks 2B is adapted to be released by an electromagnetic mechanism in the toolbox 7. In another characteristic of some embodiments, the electromagnetic mechanism works substantially similarly to that used to remove clothing tags, the electronic article surveillance (EAS) tag detacher. In another characteristic of some embodiments, if the locks 2B is released, the tip tool 1 is adapted to be removed from the fitting 2C if it is driven by manual traction or driven by another instrument.

In another characteristic of some embodiments, during the surgical procedure, each tip tool 1 is blocked in sockets 9 that it is docked.

In another characteristic of some embodiments, the tip tool 1 fixation in the sockets 9 is made by a mechanical system, magnetic or by an electromagnetic system but is not limited to them.

In another characteristic of some embodiments, in socket 9 of the toolbox 7 there are placed the necessary tip tool 1 to perform the surgery, for example: place a tweezers, or scalpel into the socket 9, but it is not limited to them.

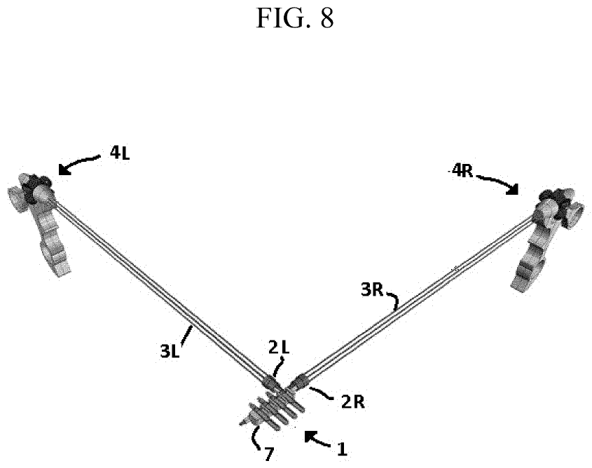

FIG. 8 is a perspective view of two video-surgical instruments, in accordance with other embodiment of the invention. Two video-surgical instruments are seen. In one characteristic of this embodiment, the adapter 2R is connected to the toolbox 7. In one characteristic of this embodiment, the adapter 2L is connected to the tip tool 1. In one characteristic of some embodiment, there are two or more tip tool 1 connected in the toolbox 7.

In another characteristic of some embodiments, to remove the tip tool 1 that is being used in the video-surgery instrument, the tip tool 1 is engaged in a free socket 9 of the toolbox 7; with the tip tool 1 positioned in the socket 9, the lock 2B is released, the tip tool 1 get stuck connected to socket 9 and the 2C assembly gets vacated.

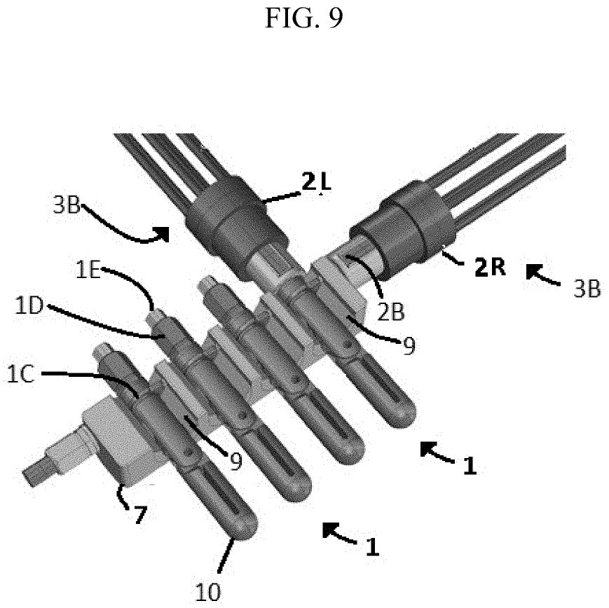

FIG. 9 is another perspective view of two video-surgical instruments showing the toolbox 7 connected to four tip tool 1, in accordance with another embodiment of the invention. Two video-surgical instruments are seen. In one characteristic of this embodiment, the adapter 2R is connected to the toolbox 7. In one characteristic of this embodiment, the adapter 2L is connected to the tip tool 1. In one characteristic of some embodiment, there are several tip tool 1 connectable to the toolbox 7.

In one characteristic of this embodiment, the invention is composed of a surgical instrument for use in video-surgery that includes a removable tip tool 1. In another characteristic of some embodiments, to facilitate operation the invention works with the aid of another video-surgical instrument specially made for this purpose, the toolbox 7.

In one characteristic of this embodiment, the surgeon chooses another tip tool 1 on another socket 9, fits the fitting 2C into the pin 1D of the chosen tip tool 1 and locks 2B. In another characteristic of some embodiments, the tip tool 1 is adapted to be removed from the fitting 2C by the surgeon's hand or pulled out longitudinally by another forceps. In some embodiments the fitting 2C and the pin 1D have hexagonal shape plus is not limited to them.

In another characteristic of some embodiments of the invention, the tip tool 1 is quickly replaced by another. In one characteristic of this embodiment, the adapter 2 is the distal end 3B of the body 3, wherein the tip tool 1 is releasably engageable. In one characteristic of this embodiment, the tip tool 1 fits into the body 3 by means of a quick-fit mechanism, usually with mechanical, magnetic and electromechanical actuation, but is not limited to them. In one characteristic of this embodiment, there are connections necessary for the mechanical, electrical and mechanical actuation of the tip tool 1 on the adapter 2 but are not limited to them. In another characteristic of some embodiments, for example: open the jaw 10, flip, angular, rotating, shoot a stapler and is not limited to them. In another characteristic of some embodiments, the handle 4 has a battery and electrical switches or buttons for activating an electrical system in the tip tool 1.

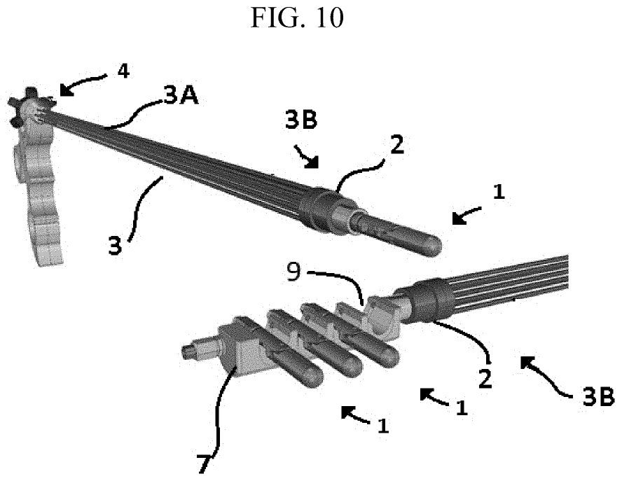

FIG. 10 is a perspective view of a video-surgical instrument that has just replaced tip tool 1 with another tip tool 1 using the toolbox 7, according to another embodiment of the invention. In one characteristic of this embodiment, there are three other tip toll 1 connected in the toolbox 7 that is adapted to be used in the video-surgical instrument.

In one characteristic of some embodiments, tip tool 1 is adapted to be: tweezers, scissors, surgical stapler, clamp, linkage mechanism, electrical system, motor, optics, solenoid, vacuum cleaner, electric scalpel, ultrasonic scissors, retractor, the toolbox 7, but is not limited to them.

In another characteristic of some embodiments, the tip tool 1 is removable by a mechanism that allows the tip tool 1 to be disconnected from the adapter 2 and another tip tool 1 be easily attached in the adapter 2. In one characteristic of this embodiment, the tip tool 1 is releasably engageable and reenactable.

In another characteristic of some embodiments the tip tool 1 is adapted to be easily replaced with another tip tool 1. For example, the tweezers may be replaced by an electric scalpel, scissors, surgical stapler or another tip tool 1.

In another characteristic of some embodiments, the tip tool 1 tends to enter by magnetic attraction in the fitting 2C of the adapter 2. In another characteristic of some embodiments, there are connections, switches, wires, necessary for mechanical, electrical and electromechanical actuation, for example: to open the claws, turn, angular, rotating, trigger the stapler, and are not limit to them. In another characteristic of some embodiments, the handle 4 has a battery and switches or electrical switches to activate an electrical system.

In another characteristic of some embodiments, the distal end 3B further including at least one articulation joint 8.

In another characteristic of some embodiments it further including an extension (do not show in the drowning) connectable do said distal end 3B and connectable to said tip tool 1. In another characteristic of some embodiments the extension further including at least one articulation joint 8.

In some embodiments the distal end 3B is adapted to interchangeably connect to a plurality of said extensions inside said surgical site. In another characteristic of some embodiments said handle 4 is adapted to be robotic operating.

In another embodiment of the invention, a method of performing a video-surgery, said method comprising the steps of: (a) inserting a video-surgical instrument inside a surgical site 11 through the trocar 5; (b) interchanging the tip tool 1 of said video-surgical instrument to another said tip tool 1 inside said surgical site 11; and (c) performing said video-surgery with said video-surgical instrument.

In one characteristic of this embodiment, the method further comprising connecting said tip tool 1 to said video-instrument inside said surgical site 11.

In one characteristic of this embodiment, the method further comprising disconnecting said tip tool 1 of said video-instrument inside said surgical site 11.

In one characteristic of this embodiment, the method further comprising inserting the toolbox 7 inside said surgical 11 site through another said trocar 5.

In one characteristic of this embodiment, the method further comprising connecting at least one said tip tool 1 to said toolbox 7.

In one characteristic of this embodiment, the method further comprising using said toolbox 7 to interchange said tip tool 1 of said video-instrument with another said tip tool 1 inside said surgical site 7.

In one characteristic of this embodiment, the method further comprising inserting another said video-surgical instrument caring one or more tip tools 1 inside said surgical site 11 through another said trocar 5.

In one characteristic of this embodiment, the method further comprising connecting an external apparatus to said video surgical instrument.

In one characteristic of this embodiment, the method further comprising interchanging said external apparatus 12 to another said external apparatus 12.

In one characteristic of this embodiment, the method further comprising interchanging said tip tool 1 to another said tip tool 1 inside said surgical site 11.

In another embodiment of the invention, a method of using the toolbox 7 in a video-surgery, said method comprising the steps of: (a) disconnect the tip tool 1 of a video-surgical instrument and connect to said toolbox 7; (b) choose another said tip tool 1 in the toolbox 7; and (c) disconnect said tip tool 1 of said toolbox 7 and connect said tip tool 1 to said surgical instrument.

In one characteristic of this embodiment, the method further comprising connecting said toolbox 7 to a video-surgical instrument.

In one characteristic of this embodiment, the method further comprising inserting said toolbox 7 connected to said video-surgical instrument in said surgical site 11 through the trocar 5.

In one characteristic of this embodiment, the method further comprising inserting at least one tip tool 1 in said toolbox 7.

In one characteristic of this embodiment, is method comprising disconnecting said toolbox 7 of said surgical instrument and connecting said toolbox 7 to another video instrument inside said surgical site.

In one characteristic of this embodiment, the method further comprising inserting another video-surgery instrument into said surgical site through another trocar 5.

In one characteristic of this embodiment, the method further comprising inserting an extension in said toolbox 7.

In one characteristic of this embodiment, the method further comprising using said toolbox 7 to connect an extension in said video-surgical instrument.

In one characteristic of this embodiment, the method further comprising removing said toolbox 7 connected to said video-surgical instrument out of said surgical site through said trocar 5.

In one characteristic of this embodiment, the method further comprising disconnecting said toolbox 7 of said video-surgical instrument.

In one characteristic of this embodiment, the method further comprising using said toolbox 7 to connect said tip tool in said surgical instrument inside said surgical site 11.

In one characteristic of this embodiment, the method further comprising connecting using said toolbox 7 to disconnect said tip tool 1 of said surgical instrument inside said surgical site 11.

In one characteristic of this embodiment, the method further comprising using said toolbox 7 to save at least one of said tip tool 1.

It will be apparent to one skilled in the art that the invention may be provided including some or all the mentioned features and components without departing from the spirit and scope of the invention. For purposes of comparing some embodiments, certain aspects and advantages of these embodiments are described. Not necessarily all such aspects or advantages are achieved by any particular embodiment. Thus, for example, some embodiments can be carried out in a manner that achieves or optimizes one advantage or group of advantages as described, herein without necessarily achieving other aspects or advantages as can also be described or suggested herein. It will also be apparent to the skilled artisan that the embodiments described above are specific examples of a broader invention which may have greater scope than any of the singular descriptions taught. There may be many alterations made in the descriptions without departing from the spirit scope of the invention. Although, certain preferred embodiments and examples are disclosed, inventive subject matter extends beyond the specifically disclosed embodiments to other alternative embodiments and/or uses, and to modifications and equivalents thereof. Thus, the scope of the claims appended hereto is not limited by any of the particular embodiments described. For example, in any method or process disclosed herein, the acts or operations of the method or process can be performed in any suitable sequence and they are not necessarily limited to any particular disclosed sequence. Some operations can be described as multiple discrete operations in turn, in a manner that can be helpful in understanding certain embodiments, however, the order of description should not be construed to imply that these operations are order dependent.

I claim:

1. An instrument for video-surgery, comprising: an elongated body; said body including a proximal end and a distal end; a tip tool connectable to said distal end; and a handle connectable to said proximal end; wherein said distal end is adapted to interchangeably connect to said tip tool inside a surgical site.

1.1. The instrument of claim 1, wherein said handle is in a functional connection to said proximal end for the transmission of at least one of force, power and torque, to said tip tool.

1.2. The surgical instrument of claim 1, wherein said handle is adapted to interchangeably connect to a plurality of external apparatus for the transmission of at least one of force, power and torque, to said tip tool.

1.3. The instrument of claim 1, further including an extension connectable do said distal end and connectable to said tip tool.

1.4. The instrument of claim 1, further including at least one articulation joint.

1.5. The instrument of claim 1, wherein said handle is adapted to be robotic operating.

1.6. The instrument of claim 1, wherein said tip tool is a toolbox.

1.7. The instrument of claim 1, wherein said tip tool comprises a pair of cooperating jaws made to perform a surgical operation.

1.8. The instrument of claim 1, wherein said tip tool is rotatable around one or more axes.

2. A method of performing a video-surgery, said method comprising the steps of:
a) inserting a video-surgical instrument inside a surgical site through a trocar;
b) interchanging a tip tool of said video-surgical instrument to another said tip tool inside said surgical site; and
c) performing said video-surgery with said video-surgical instrument.

2.1. The method of claim 2, further comprising inserting a toolbox inside said surgical site through another said trocar.

2.2. The method of claim 2, further comprising connecting at least one said tip tool in said toolbox.

2.3. The method of claim 2, further comprising using said toolbox to interchange said tip tool of said video-instrument with another said tip tool inside said surgical site.

2.4. The method of claim 2, further comprising inserting another said video-surgical instrument caring one or more tip tools inside said surgical site through another said trocar;

2.5. The method in claim 2, further comprising interchanging an external apparatus to another said external apparatus;

3. A method of using a toolbox in a video-surgery, said method comprising the steps of:
a) disconnect a tip tool of a video-surgical instrument and connect to said toolbox;
b) choose another said tip tool in said toolbox; and
c) disconnect said tip tool of said toolbox and connect said tip tool to said surgical instrument.

3.1. The method of claim 3, further comprising connecting said toolbox to a video-surgical instrument.

3.2. The method of claim 3, further comprising inserting said toolbox connected to said video-surgical instrument in said surgical site through a trocar

3.3. The method of claim 3, further comprising disconnecting said toolbox of said surgical instrument and connecting said toolbox to another video instrument inside said surgical site.

3.4. The method of claim 3, further comprising using said toolbox to connect said tip tool in said surgical instrument inside said surgical site.

3.5. The method of claim 3, further comprising using said toolbox to disconnect said tip tool of said surgical instrument inside said surgical site.

3.6. The method of claim 3, further comprising using said toolbox to save at least one of said tip tool.
